(19) United States
(12) Patent Application Publication
(10) Pub. No.: US 2005/0209559 A1
(43) Pub. Date: Sep. 22, 2005

(54) Apparatus and methods for the treatment of chronic total occlusions

(75) Inventors: Ronan Thornton, Galway (IE); Anthony Bruce Semedo, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc. A Delaware Corporation, Santa Rosa, CA (US)

(21) Appl. No.: 11/084,842

(22) Filed: Mar. 18, 2005

Related U.S. Application Data
(60) Provisional application No. 60/554,876, filed on Mar. 19, 2004.

(51) Int. Cl. A61M 29/00

(52) U.S. Cl. 604/103.04

(57) ABSTRACT

According to a first aspect of the invention there is provided a catheter assembly. A lumen extends through the catheter assembly and terminates with an exit port. The lumen being configured to receive a guide wire therein. The catheter assembly comprises a flexible distal segment having a predetermined length and a first predetermined outer diameter, a proximal segment having a second outer diameter greater than that of the first outer diameter, and an intermediate tapered section between the first proximal segment and the distal segment.

### RELATED APPLICATIONS

This application claims the benefit of U.S. Provisional Patent Application 60/554,876 filed Mar. 19, 2004.

### FIELD OF THE INVENTION

This invention relates generally to intra-luminal devices for the treatment of stenotic or diseased lumens, and more particularly, to a catheter assembly and method for the treatment of chronic total occlusions (CTO) in a lumen.

### BACKGROUND OF THE INVENTION

Stenotic lesions may comprise a hard, calcified substance and/or a softer thrombus material, each of which forms on the lumen walls of a blood vessel and restricts blood flow therethrough. Intra-luminal treatments such as balloon angioplasty, stent deployment, atherectomy, and thrombectomy are well known and have proven effective in the treatment of such stenotic lesions. These treatments often involve the insertion of a therapy catheter into a patient's vasculature which may be torturous and may have numerous stenoses of varying degrees throughout its length. In order to place the distal end of a catheter at the treatment site, a guidewire is typically introduced and tracked from an incision, through the stenosis, and across the lesion. Then, a catheter (e.g. a balloon catheter), perhaps containing a stent at its distal end can be tracked over the guidewire to the treatment site. Ordinarily, the distal end of the guidewire is quite flexible so that as it is pushed through the lumen, it can find its way through the turns of the typically irregular passageway without damaging the lumen.

In some instances, the extent of occlusion of the lumen is so severe that the lumen is completely or nearly completely obstructed, leaving virtually no passageway for the guidewire. Such a condition may be described as a total occlusion. If this occlusion persists for a long period of time, the lesion is referred to as a chronic total occlusion or CTO. Furthermore, in the case of diseased blood vessels, the lining of the vessels may be characterized by the prevalence of atheromatous plaque, which may form total occlusions. The extensive plaque formation of a chronic total occlusion typically has a fibrous cap surrounding softer plaque material. This fibrous cap may present a surface that is difficult to penetrate with a conventional guidewire, and the typically flexible distal tip of the guidewire may be unable to cross the lesion. In such cases, a recanalizing device such as a stiffer guidewire may be employed to traverse the stenosis. In such cases, additional precautions must be taken to prevent injury to the vessel wall. For example, it is imperative that the guidewire or other recanalizing device be centered within the vessel to avoid penetration of the vessel wall.

Accordingly, it would be desirable to provide an improved intraluminal device or catheter assembly for use in the treatment of chronic total occlusions, the device being capable of penetrating the proximal cap of the occlusion, finding and navigating through the true lumen, and exiting the distal cap of the occlusion.

### BRIEF SUMMARY OF THE INVENTION

According to a first aspect of the invention there is provided an improved catheter assembly. A lumen extends through the catheter assembly and terminates with an exit port. The lumen is configured to receive a guidewire therein. The catheter assembly comprises a flexible distal segment having a predetermined length and a first predetermined outer diameter, a proximal segment having a second outer diameter greater than that the first outer diameter, and an intermediate tapered section between the flexible proximal segment and the distal segment.

According to a further aspect of the invention there is provided a catheter assembly comprising a first flexible tube having a lumen therethrough and having a first distal exit port configured to receive a guidewire therein. The first flexible tubular member may include blades or ribs on a distal outer surface. A second flexible tube has a second distal exit port and is configured to slidingly receive the first flexible tube therein. An expandable membrane is coupled proximate a distal end of the second flexible tube for substantially centering the guidewire when the membrane is expanded.

### BRIEF DESCRIPTION OF THE DRAWINGS

The present invention will hereinafter be described in conjunction with the following drawing figures, wherein like numerals denote like elements, and

FIGS. 1 and 2 are cross-sectional views illustrating potential problems associated with the treatment of chronic total occlusions;

FIG. 3 illustrates a guiding catheter assembly positioned within a patient's vasculature;

FIG. 4 is a cross-sectional view of a guidewire catheter in accordance with a first embodiment of the present invention;

FIGS. 5 and 6 illustrates the use of steering elements or wires in the catheter shown in FIG. 4;

FIG. 7 illustrates the use of four steering elements or wires in the catheter shown in FIG. 4 so as to permit steering in two planes;

FIG. 8 illustrates how the guidewire shown in FIG. 4 may be utilized to open a channel in a total occlusion;

FIGS. 9, 10 and 11 are cross-sectional views of yet another embodiment of the present invention.

### DETAILED DESCRIPTION OF THE INVENTION

The following detailed description is merely exemplary in nature and is not intended to limit the invention or the application and use of the invention. Furthermore, there is no intention to be bound by any expressed or implied theory presented in the preceding technical field, background, brief summary or the following detailed description.

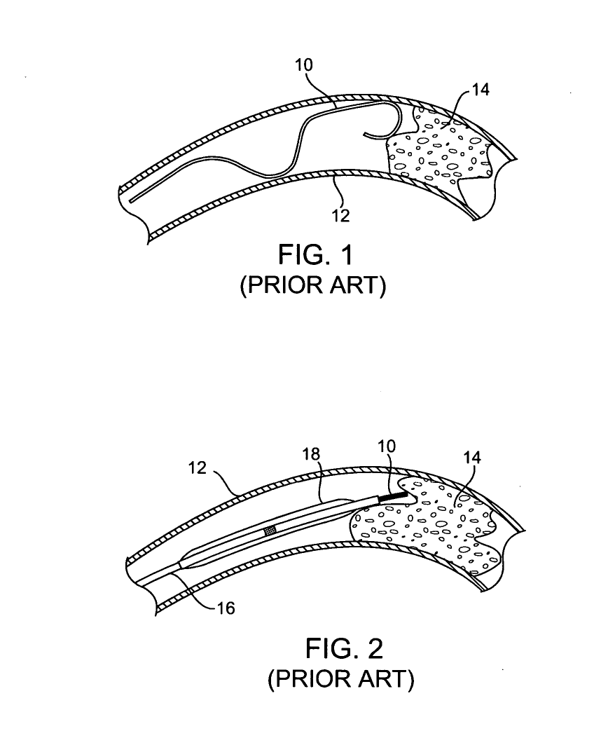

FIGS. 1 and 2 are cross-sectional views illustrating potential problems associated with the treatment of chronic total occlusions. Referring to FIG. 1, a standard guidewire 10 is advanced through a vessel 12 via a catheter (not shown) to the site of a chronic total occlusion 14. As depicted in FIG. 1, guide wire 10 may be unable to penetrate the proximal cap of occlusion 14 and may prolapse into vessel 12 when pressure is applied.

FIG. 2 illustrates a prior art catheter 16 having a balloon 18 mounted thereon and the limitations of such when attempting to center a device such as guidewire 10 at the site of chronic total occlusion 14. As can be seen, guidewire 10 is not directed toward the center of occlusion 14, but in fact is undesirably directed toward the wall of vessel 12. Thus, difficulties will be encountered during attempts to traverse occlusion 14, and the risk of perforating vessel 12 is high. It should be appreciated that in the arrangement shown in FIG. 2, catheter 16 confers no advantage with regard to the centering of guidewire 10.

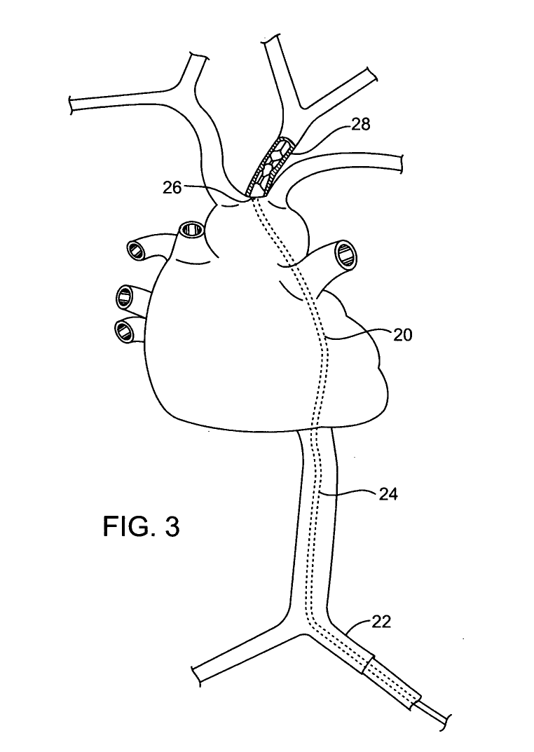

Referring to FIG. 3, a guiding catheter assembly 20 is shown positioned within a patient's vasculature. Typically, the guiding catheter assembly 20 is first inserted through an incision (not shown) and into a femoral artery 22 of a patient. The assembly 20 is then advanced through artery 22 into the patient's aorta 24 and then into the ostium 26 of the selected carotid artery or vessel; for example, the left carotid 28. Guiding catheter assembly 20 is positioned by a physician, preferably proximal to the stenotic lesion or occlusion.

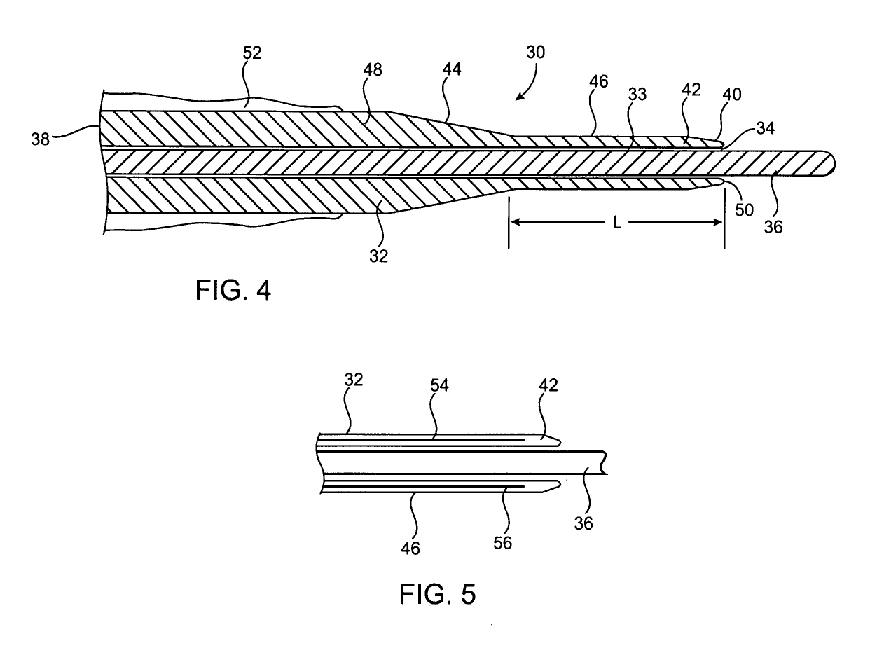

FIG. 4 is a cross-sectional view of a guidewire catheter assembly in accordance with a first embodiment of the present invention. Catheter assembly 30 comprises a catheter 32 having a guide wire lumen 33 therethrough and terminating at exit port 34. Guidewire 36 may be telescopically inserted into guidewire lumen 33 in the well known manner. As can be seen, catheter assembly 30 has a proximal end 38 and a distal end 40. Catheter 32 includes a distal segment 46, a proximal segment 48, and an intermediate tapered section 44. Tapered region 44 acts as a transition between distal region 46 and a proximal region 48. An additional tapered segment 42 is provided at the distal end of distal segment 46.

As can be seen, the inner diameter of catheter 32, in this embodiment, is substantially constant (0.016 inches) throughout its length whereas the outer diameter of proximal region 48 is significantly larger than that of outer region 46. For example, proximal segment 48 may have an outer diameter of approximately 0.030 inches, and distal segment 46 may have an outer diameter of 0.022 inches. Thus, distal region 46 provides a low profile flexible segment having a length of, for example, 5 to 15 millimeters, that may be advanced into a lesion with minimum trauma and facilitates steering of wire 36 in the lesion. The larger proximal section 48 may be pushed through the lesion after the wire to dilate the lesion in preparation for the introduction of, for example, a balloon. The distal portion of proximal tip 40 may be provided with a radiopaque substance 50 such as barium sulfate without compromising tracking profile.

As stated previously, the inner diameter of wire lumen 33 is relatively constant along the length of catheter 32. However, it may be desirable to increase the inner diameter of proximal region 48, for example to 0.022 inches-0.024 inches, in order to improve wire movement and facilitate the injection of dye through wire lumen 33. Furthermore, a standard PTCA balloon 52 may be added. The distal tip can be tracked into the lesion, and when through the lesion, balloon 52 can be brought into the lesion to open the path for further dilation.

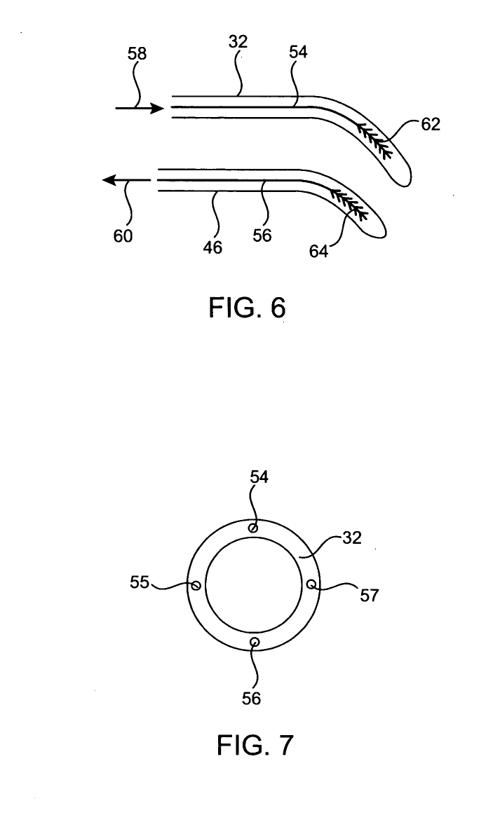

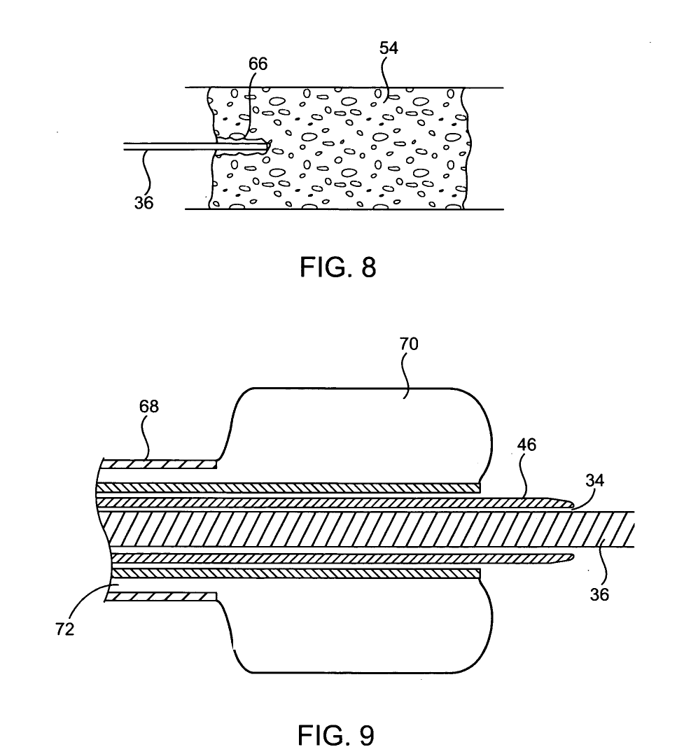

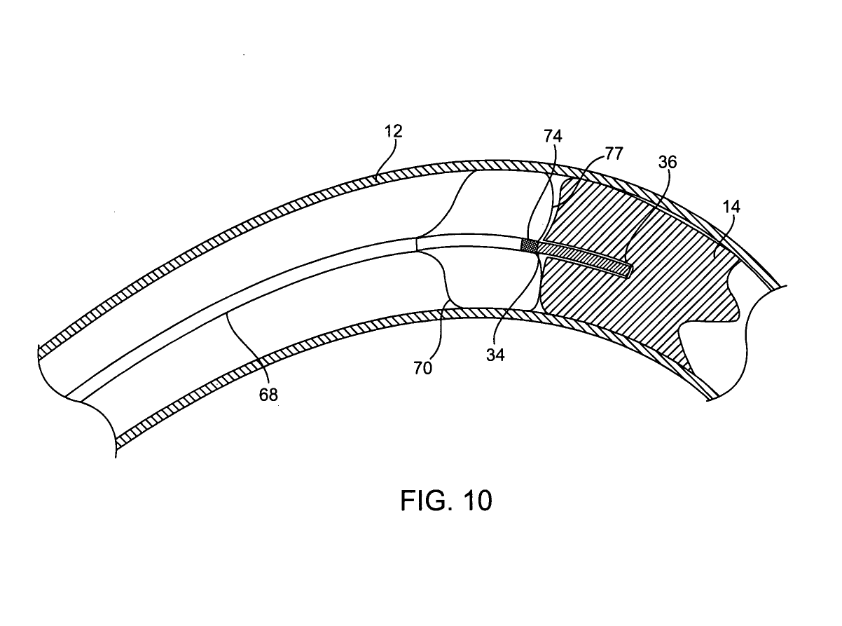

If desired, steering elements (e.g. wires 54 and 56) may be imbedded into lumen shaft 32 to provide a steering capability at the distal end of the shaft 40 as is shown in FIG. 5. Wires 54 and 56 may be anchored at the distal end thus enabling the distal tip of the shaft to be deflected as is shown in FIG. 6. That is, if wires 54 and 56 are anchored at their distal ends as is shown at 62 and 64 respectively, pushing wire 54 at its proximal end as is indicated by arrow 58 and/or pulling wire 56 at its proximal end as is indicated by arrow 60 will cause the distal tip of the catheter to bend. It should be understood that while FIGS. 5 and 6 illustrates the use of two steering wires 54 and 56, four steering wires could be employed to provide steering in two planes instead of a single plane as is shown in FIG. 7. These steering elements facilitate both the positioning of the guidewire in the proximal cap and the further steering of the guidewire through the lesion. Furthermore, by repeatedly flexing the tip of the guidewire in, for example, a back and forth motion, it could be used to locally open a micro-channel as is shown in FIG. 8.

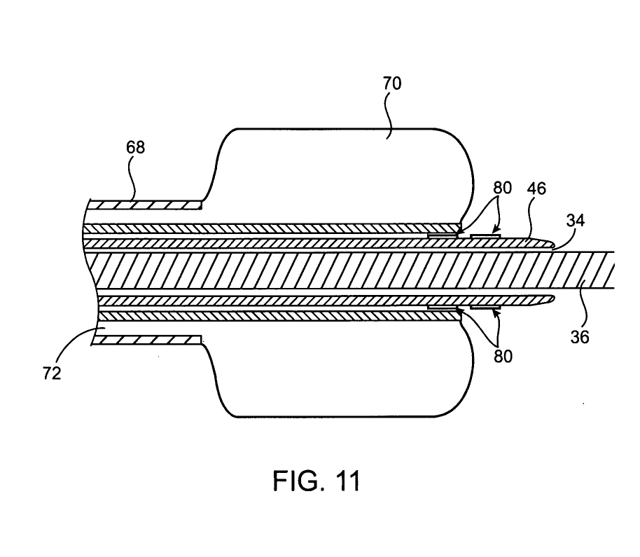

FIGS. 9 and 10 illustrate yet another embodiment of the present invention which substantially reduce the problems described in connection with FIGS. 1 and 2 above. As can be seen, flexible tubular guidewire catheter 46 acts as an inner shaft which is slidably mounted within an outer flexible tubular balloon-catheter 68 equipped with an expandable membrane (e.g. a compliant balloon) 70. Inner shaft 46 may further include blades or ribs 80 mounted on a distal section as shown in FIG. 11. Blades 80 are housed within outer shaft 68 to protect the vessel from damage until inner shaft 46 is advanced. Sliding inner shaft 46 is advanced over wire 36 and into chronic total occlusion 14 in order to score the lesion and potentially facilitate controlled expansion or opening of the lesion. As can be seen, balloon 70 may be inflated by means of inflation channel 72. Inner shaft 46 may have an outer diameter of 0.023 inches and an inner diameter of 0.016 inches. Outer shaft 68 may have an inner diameter of 0.041 inches and an outer diameter of 0.047 inches.

Compliant balloon 70, when inflated or expanded, serves to center wire lumen 46 and therefore guidewire 36 within the vessel to be treated. For example, referring to FIG. 10, balloon 70 centers exit port 34 thereby centering guidewire 36 within vessel 12. Distal portion 77 of balloon 70 substantially abuts at least a portion of chronic total occlusion 14. If desired, catheter 68 may be provided with a marker band 34 on its distal portion. Therefore, marker band 34 also substantially abuts chronic total occlusion 14 and enables a clinician to predict the location of the central region of chronic total occlusion 14. Subsequently, guidewire 36 may be replaced by an alternative tool designed, for example, to penetrate occlusion 14 or, for example, perform a balloon angioplasty and/or stent delivery procedure. In a further example, after sliding catheter shaft 46 is retracted into outer shaft 68, a balloon catheter can then be advanced into the scored lesion and inflated to dilate the lesion. Ideally, a single device can be used to support wire crossing of the lesion, score the lesion surface and dilate the lesion. Any such device will also be centered within vessel 12 by means of balloon 70. For a more complete discussion, the interested reader is directed to U.S. Pat. No. 6,533,753 issued Mar. 18, 2003 and entitled “APPARATUS AND METHOD FOR TREATMENT OF AN OCCLUDED LUMEN”.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient roadmap for implementing an exemplary embodiment of the invention, it being understood that various changes may be made in the function and arrangement of elements described in an exemplary embodiment without departing from the scope of the invention as set forth in the appended claims.

1. A catheter assembly having a lumen therethrough terminating the exit port, said lumen configured to receive a guide wire therein, said catheter comprising:
a flexible distal segment having a predetermined length and a first predetermined outer diameter;
a proximal segment having a second outer diameter greater than said first outer diameter; and
an intermediate tapered section between said proximal segment and said distal segment.

2. An assembly according to claim 1 wherein said distal segment has a tapered distal end.

3. An assembly according to claim 2 wherein said predetermined length is approximately five to fifteen millimeters.

4. An assembly according to claim 2 further comprising a first steering element in said catheter assembly.

5. An assembly according to claim 4 further comprising at least a second steering element in said catheter assembly so as to permit steering of said catheter assembly in a first plane.

6. An assembly according to claim 5 further comprising at least third and fourth steering elements in said catheter assembly to permit steering of said catheter assembly in a second plane.

7. An assembly according to claim 5 wherein said first and second steering elements comprise first and second wires.

8. An assembly according to claim 7 wherein each of said first and second steering wires has an anchored distal end.

9. An assembly according to claim 8 further comprising a balloon mounted on said catheter assembly.

10. An assembly according to claim 4 wherein said lumen has a substantially constant inner diameter.

11. An assembly according to claim 4 wherein said lumen has a larger inner diameter in said proximal segment than in said distal segment.

12. A catheter assembly, comprising:
a first flexible tube having a lumen therethrough and a first exit port configured to receive a guide wire therein;
a second flexible tube a second distal exit port configured to slidingly receive said first flexible tube therein; and
an expandable membrane coupled proximate distal end of said second flexible tube for substantially centering said guide wire when said membrane is expanded.

13. An assembly according to claim 12 wherein said expandable membrane is a balloon.

14. An assembly according to claim 12 wherein the first flexible tube has a distal outer section with blades.
